(19) United States
(12) Patent Application Publication
(10) Pub. No.: US 2017/0258448 A1
(43) Pub. Date: Sep. 14, 2017

(54) ULTRASONIC TRANSDUCER UNIT, AND INFORMATION ACQUISITION APPARATUS INCLUDING THE ULTRASONIC TRANSDUCER UNIT

(71) Applicant: CANON KABUSHIKI KAISHA, Tokyo (JP)

(72) Inventors: Ayako Maruyama, Sagamihara-shi (JP); Kazutoshi Torashima, Yokohama-shi (JP); Atsushi Kandori, Ebina-shi (JP)

(21) Appl. No.: 15/450,529

(22) Filed: Mar. 6, 2017

(30) Foreign Application Priority Data
Mar. 8, 2016 (JP) 2016-045038

(51) Int. Cl. A61B 8/00; A61B 8/08; A61B 5/00

(52) U.S. Cl. CPC A61B 8/4494; A61B 5/0095; A61B 8/5207

(57) ABSTRACT

The technology concerning improvement of resolution of an acoustic wave sensor having a hemispherical shape or the like is provided. An ultrasonic transducer unit includes an ultrasonic transducer having a plurality of ultrasonic transducer elements, and a probe casing configured to support a plurality of the ultrasonic transducers, and to have a concave portion facing a subject. The plurality of ultrasonic transducer elements is arranged on a same plane facing a center of curvature of the probe casing. The plurality of ultrasonic transducer elements is arranged in a rotationally symmetrical manner about a normal line connecting the center of curvature of the probe casing to a point on a plane of the ultrasonic transducer.

### BACKGROUND

Field

Aspects of the present invention generally relate to an ultrasonic transducer unit including an ultrasonic transducer, such as a capacitive transducer, an information acquisition apparatus, such as a photoacoustic apparatus, provided with the ultrasonic transducer, and the like.

Description of the Related Art

Conventionally, in fabrication of micromachine members using micromachining techniques, processing in a micrometer order is possible, and a variety of minute function elements has been provided using such micromachine members. A capacitive micromachined ultrasonic transducer (CMUT) fabricated using the micromachining techniques has been researched as a substitute of a piezoelectric element. Such a CMUT can transmit and receive ultrasonic waves, and the like, using vibration of a vibration film. Particularly, the CMUT exhibits excellent broadband characteristics when being used in liquid.

With respect to those techniques, U. S. Patent Publication No. 2011/0306865 discloses an apparatus including a hemispherical acoustic wave sensor on which a plurality of ultrasonic transducers is arranged in a hemispherical manner, and a cup-shaped container in which a region of a subject to be inspected is to be set.

Since it is difficult to mount ultrasonic transducer elements to a hemispherical curved surface with high density, there has been proposed a mounting method in which an assembly or a group (hereinafter referred to an ultrasonic transducer) of a plurality of ultrasonic transducer elements arranged on the same plane is prepared, and the assemblies are mounted. In such a method, if all the ultrasonic transducer elements are arranged in a manner so as to face a center of curvature of the hemispherical acoustic wave sensor, there is a possibility that ability of detecting signals generated at positions other than the center of curvature decreases. In a case where an inspection target region is present at the center of curvature and around the center of curvature, uneven distribution of image resolution occurs in the inspection target region. Hence, an image quality is likely to be degraded.

### SUMMARY

According to an aspect of the present invention, an ultrasonic transducer unit includes an ultrasonic transducer including a plurality of ultrasonic transducer elements, and a probe casing configured to support the plurality of ultrasonic transducers, and to have a concave portion facing a subject to be located at a predetermined position. The plurality of ultrasonic transducer elements is arranged on a same plane facing a center of curvature of the probe casing. Further, the plurality of ultrasonic transducer elements is arranged in a rotationally symmetrical manner about a normal line connecting the center of curvature of the probe casing to a point on a plane of the ultrasonic transducer.

Further features of aspects of the present invention will become apparent from the following description of exemplary embodiments with reference to the attached drawings.

### BRIEF DESCRIPTION OF THE DRAWINGS

FIGS. 1A to 1C are diagrams illustrating an example of a photoacoustic diagnosis apparatus according to the present invention, and an example of an ultrasonic transducer unit in the photoacoustic diagnosis apparatus.

FIGS. 2A to 2C are diagrams each illustrating an example of an ultrasonic transducer.

FIG. 3A is a diagram illustrating an example of an ultrasonic transducer, and FIG. 3B is a graph illustrating a decreasing ratio of sensitivity due to directionality of an element.

FIG. 4A is a diagram illustrating an example of an ultrasonic transducer, and FIG. 4B is a graph illustrating a decreasing ratio of sensitivity due to directionality of an element.

FIGS. 5A to 5C are diagrams illustrating an example of an ultrasonic transducer according to a first exemplary embodiment.

FIGS. 6A to 6C are diagrams illustrating an ultrasonic transducer, an element, and a cell, respectively, according to the first exemplary embodiment.

FIGS. 7A to 7E are diagrams illustrating an example of a fabrication method of a capacitive transducer element according to the first exemplary embodiment.

FIGS. 8A to 8C are diagrams illustrating a casing, a transducer, and a cell, respectively, according to the first exemplary embodiment.

FIG. 9 is a diagram illustrating a receiving preamplifier connected at a stage following an element.

FIGS. 10A to 10C are graphs illustrating overall receiving sensitivity, an output current, and a current/voltage conversion gain of the receiving preamplifier, respectively.

FIGS. 11A to 11C are graphs illustrating overall receiving sensitivities of element groups according to the first exemplary embodiment and a second exemplary embodiment.

FIGS. 12A and 12B are graphs illustrating overall receiving sensitivities of an element group according to a third exemplary embodiment.

FIGS. 13A and 13B are graphs illustrating overall receiving sensitivities of an element group according to a fourth exemplary embodiment.

### DESCRIPTION OF THE EMBODIMENTS

One aspect of the present invention has the following features. Namely, in order to improve resolution of an acoustic wave sensor having a hemispherical shape or the like, a plurality of ultrasonic transducers is arranged in a manner so as to face a center of curvature of a concave probe casing. On each of the plurality of ultrasonic transducers, a plurality of ultrasonic transducer elements (hereinafter also referred to as elements) is arranged on the same plane, in a rotationally symmetric manner about a normal line connecting the center of curvature to a point on the plane. In this structure, the ultrasonic transducer elements on each of the plurality of ultrasonic transducers are directed in the same direction on the same plane. More specifically, normal lines extending perpendicularly from each of the elements are approximately parallel with each other. Accordingly, when an ultrasonic transducer is arranged in such a manner that a center element of the elements faces the center of curvature or a normal line extending from the center element is a perpendicular line extending from the center of curvature, normal lines extending from other elements disposed around the center element pass through points off from the center of curvature. By using such a feature, it is possible to improve distribution of resolution in a region of a subject. The distribution of resolution can be further improved by adjusting receiving sensitivity characteristics of each of the ultrasonic transducer elements (characteristics obtained in consideration of gain characteristics of an amplifier and the like connected at the stage following the element, in addition to output characteristics of the element) according to an extension of the subject.

Exemplary embodiments according to the present invention will be described below with reference to the drawings. In the description, elements having the same configuration are in principle referred to by the same reference numeral, and repeated description is omitted or simplified. The following calculation formulae, calculation methods, materials, sizes, shapes, and the like should be appropriately modified according to configurations of an apparatus and various conditions to which aspects of the present invention are applied, and it should not be understood that a scope of the present invention is limited to the following description.

### <System Configuration>

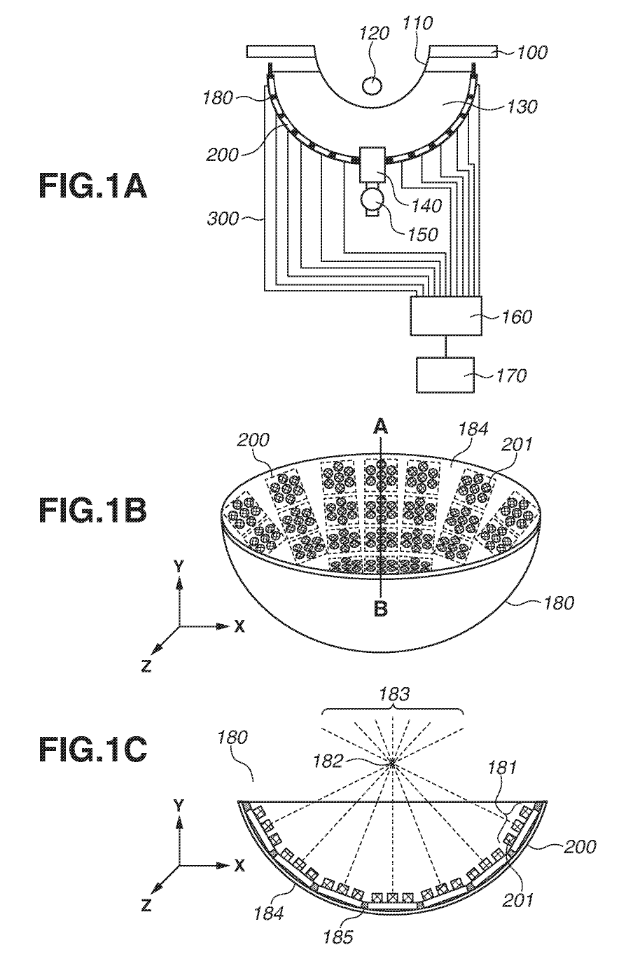

With reference to FIGS. 1A to 1C, a configuration example of an ultrasonic transducer unit (hereinafter also referred to as an ultrasonic unit) and a photoacoustic diagnosis apparatus (hereinafter also referred to as an information acquisition apparatus) according to an exemplary embodiment of the present invention will be described. FIG. 1A is a diagram illustrating an overall configuration of the present exemplary embodiment. The photoacoustic diagnosis apparatus according to the present exemplary embodiment includes a mounting portion 100, a shape retaining unit 110 for retaining a subject 120, an acoustic matching material 130, an optical system 140, a light source 150, a processing portion 160, an image display portion 170, and an ultrasonic unit 180. A breast or the like of an examinee (the subject 120) is inserted into the shape retaining unit 110 and measurement is executed. Pulse light from the light source 150 is directed toward the shape retaining unit 110 from a vicinity of an apex of the ultrasonic unit 180 having a concave shape through the optical system 140 that is a part of a light irradiation portion for irradiating the subject 120 with light. Thus, the subject 120 is irradiated with light. When a part of energy of light transmitting in the subject 120 is absorbed by an optical absorber, such as blood, an acoustic wave is generated due to thermal expansion of the optical absorber in the subject 120. The acoustic wave generated in the subject 120 transmits in all directions, and passes through the acoustic matching material 130. Then, the acoustic wave is received by each ultrasonic transducer 200 arranged in the ultrasonic unit 180, and analyzed by the processing portion 160. A result of the analysis is output to the image display portion 170 as an image that represents characteristic information of the subject 120.

### <Light Source>

The light source 150 is an apparatus that emits the pulse light. The light source 150 is desirably a laser light source. A light emitting diode, a flash lamp, or the like can also be used. An irradiation time point, a waveform, intensity, and the like of the pulse light are controlled by a light source control portion (not illustrated). The light should be emitted at a sufficiently short time period according to thermal characteristics of the subject 120 such that the photoacoustic wave can be effectively generated. In a case where the subject 120 is a living body, it is desirable that a pulse width of the pulse light from the light source 150 is approximately 50 nanoseconds. A wavelength of the pulse light is desirably a wavelength that enables the light to be transmitted into an inner portion of the subject 120. Specifically, approximately a range between 600 nm or more and 1200 nm or less is desirable. The light in such a wavelength range can reach a comparatively deep portion of the living body, and information of the deep portion can be obtained. Further, the wavelength of the pulse light having a large absorption factor for a measurement object is desirable.

### <Optical System>

The optical system 140 is a unit for guiding the pulse light from the light source 150 to the subject 120. Specifically, the optical system 140 is an optical member including an optical fiber, a lens, a mirror, a diffusion plate, or the like such that a desired beam shape and light intensity distribution can be obtained. Further, when the light is guided, the shape and density of the light can be modified by using the above optical members to achieve a desired light distribution. The optical members are not limited to the above-described ones. The optical member can be any one so far as the above function is satisfied.

### <Shape Retaining Unit>

The shape retaining unit 110 is a member for retaining the shape of the subject 120 constant. The shape retaining unit 110 is mounted to the mounting portion 100. In a case where the subject 120 is irradiated with the light through the shape retaining unit 110, it is desirable that the shape retaining unit 110 is transparent to the irradiation light. For example, polymethylpentene, polyethylene terephthalate, or the like can be used as material of the shape retaining unit 110. When the subject 120 is a breast, in order to retain the shape of the breast constant in a way so that deformation is reduced, a shape of the shape retaining unit 110 is desirably a shape formed by cutting a sphere along a certain cross section (a partial spherical shape), or the like. A shape of the shape retaining unit 110 can be appropriately designed according to a cubic content of the subject 120 and a desired shape of the subject 120 retained. The shape retaining unit 110 is desirably formed such that the shape retaining unit 110 fits to an external form of the subject 120, and that the shape of the subject 120 is approximately the same as the shape of the shape retaining unit 110. Further, the photoacoustic diagnosis apparatus can carry out the measurement without using the shape retaining unit 110.

### <Subject>

The subject 120 is a measurement target. For example, the subject 120 is a breast of a living body, or the like. When the photoacoustic diagnosis apparatus is adjusted, a phantom imitating acoustic characteristics and optical characteristics of the living body can be used.

### <Acoustic Matching Material>

The acoustic matching material 130 is a material with which a space between the subject 120 and the ultrasonic unit 180 is filled to connect the subject 120 with the ultrasonic unit 180 acoustically. In the present exemplary embodiment, the acoustic matching material 130 can be interposed between the ultrasonic unit 180 and the shape retaining unit 110. Further, the acoustic matching material 130 is also interposed between the shape retaining unit 110 and the subject 120. The acoustic matching material 130 interposed between the ultrasonic unit 180 and the shape retaining unit 110 can be different from the acoustic matching material 130 interposed between the shape retaining unit 110 and the subject 120.

The acoustic matching material 130 is desirably a material in which the photoacoustic wave is hard to be attenuated. Further, the acoustic matching material 130 is desirably a material through which the pulse light from the light source 150 is transmitted. In addition, the acoustic matching material 130 is desirably a liquid. Specifically, water, a castor oil, a gel, or the like can be used as the acoustic matching material 130.

### <Ultrasonic Transducer Unit>

The ultrasonic unit 180 is a unit for converting the acoustic wave generated in the subject 120 into an analog electrical detection signal. The ultrasonic unit 180 has a concave portion which is concave toward the subject 120 disposed at a measurement position. In the present exemplary embodiment, the ultrasonic unit 180 has a cup shape that is approximately hemispherical or partially spherical. A radius of the concave portion on a side of the subject 120 is, for example, from several millimeters to several tens centimeters. The radius can be changed according to a size of the subject 120. Further, a thickness of the concave portion, that is, a difference between a radius of a surface (an inner surface) of the concave portion on the side of the subject and a radius of a surface (an outer surface) of the concave portion on the other side of the subject, is, for example, from several millimeters to several centimeters. The thickness can be changed according to an overall size of the apparatus. The ultrasonic unit 180 is mounted to the mounting portion 100 in a position adjustable manner.

FIG. 1B is a perspective view illustrating the ultrasonic unit 180. A plurality of the ultrasonic transducers 200 is arranged in the ultrasonic unit 180. The ultrasonic transducer 200 includes a plurality of ultrasonic transducer elements 201 (i.e., an element group 210 described below). The ultrasonic transducer element 201 can be of any type so far as it can receive the ultrasonic wave. The element 201 can be of a type that can receive and transmit a ultrasonic wave. For example, a piezoelectric ceramic material, such as lead zirconate titanate (PZT), a polymer piezoelectric film material, such as PolyVinylidene DiFluoride (PVDF), or the like can be used. Further, an element other than the piezoelectric element can also be used. For example, a capacitive element, such as CMUT, an acoustic wave receiving element using a Fabry-Perot interferometer, or the like can be used. A sealing member 185 (see FIG. 1C) is desirably interposed between the ultrasonic transducer 200 and a casing 184 (described below) to prevent infiltration of the acoustic matching material into a space between the ultrasonic transducer 200 and the casing 184.

### <Ultrasonic Transducer>

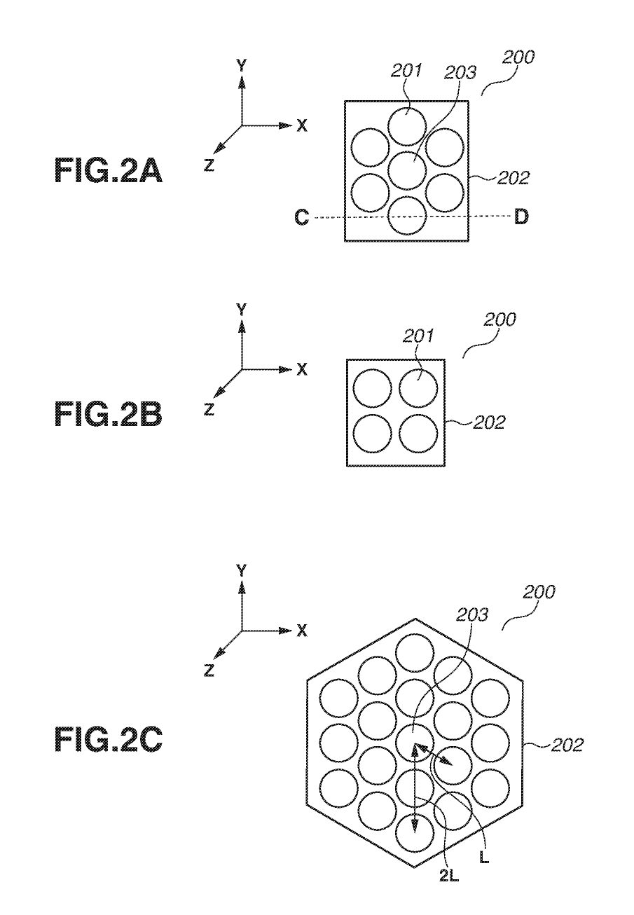

A configuration of the ultrasonic unit 180 and the ultrasonic transducer 200 will be described. FIG. 1B is a view illustrating an example of the ultrasonic unit 180 in the photoacoustic diagnosis apparatus. FIG. 1C is a cross sectional view taken along a line A-B in FIG. 1B. FIGS. 2A to 2C are top views each illustrating an example of the ultrasonic transducer 200.

The ultrasonic transducers 200 supported by the casing 184 which is a probe casing are arranged in a manner so that each sensor plane 181 faces the side of the subject 120. More specifically, the sensor plane 181 is arranged so as to face approximately a center 182 of curvature of the casing 184 which is a prove casing having a concave shape according to FIG. 1C. The ultrasonic transducer 200 is connected to the processing portion 160 through a wiring line 300, such as a conductive line and a cable. Material of the casing 184 in the ultrasonic unit 180 can be any one that can be formed into a concave shape, such as an approximately hemispherical shape. The material is, for example, a metal, ceramics, or resin. According to FIG. 1C, there are seven (7) normal lines 183 each of which is formed by connecting the center 182 of curvature of the casing 184 to a predetermined point (for example, a center) on the sensor plane 181 of each ultrasonic transducer 200. In the ultrasonic transducer 200, a plurality of the ultrasonic transducer elements 201 is arranged in a rotationally symmetrical manner about the normal line 183 connecting the center 182 of curvature of the casing 184 to the point on the sensor plane 181 of the ultrasonic transducer 200. In the rotationally symmetrical arrangement, an arrangement of the ultrasonic transducer elements 201 when the plane 181 is rotated about the normal line by a predetermined angle excluding 360 degrees remains unchanged from its original arrangement. In the following example, the arrangement of the ultrasonic transducer elements 201 every time when the plane is rotated at an equi-angular interval (for example, 60 degrees or 90 degrees) remains unchanged from its original arrangement. Further, the ultrasonic transducer elements 201 are arranged around the normal line with approximately equal density.

FIG. 2A is a top view illustrating an example of the ultrasonic transducer 200. In this example, seven (7) ultrasonic transducer elements 201 are arranged in a single ultrasonic transducer 200. When the ultrasonic transducer 200 is mounted to the casing 184, a center element 203 of the seven ultrasonic transducer elements 201 faces approximately the center 182 of curvature of the casing 184. Among the seven ultrasonic transducer elements 201, six (6) ultrasonic transducer elements 201 surrounding the center element 203 are arranged at an equi-angular interval about the normal line 183 connecting the center 182 of curvature of the casing 184 to the point on the sensor plane 181. The number of the elements 201 is not limited to seven, and a desired number of the elements 201 can be arranged. The following arrangement can also be adopted. Namely, in one arrangement as illustrated in FIG. 2B, the center element 203 facing the center 182 of curvature is omitted. Further, in another arrangement illustrated in FIG. 2C, the center element 203 is disposed so as to face the center 182 of curvature of the casing 184, and nineteen (19) ultrasonic transducer elements are arranged in more than one concentric circles. An external configuration 202 of the ultrasonic transducer 200 can be formed in a desired shape, such as an approximately circular shape and a polygonal shape.

With respect to various arrangement examples of the ultrasonic transducer elements 201, the characteristics of each arrangement will be described. For example, in a case where the subject 120 extends broader than the center 182 of curvature of the casing 184, sensitivity characteristics of seven (7) ultrasonic transducer elements 201 in the ultrasonic transducer 200 illustrated in FIG. 2A are preferably designed to be equal to each other. The center element 203 can detect the acoustic wave from the center 182 of curvature of the casing 184 with high sensitivity. In contrast to the center element 203, the ultrasonic transducer elements 201 surrounding the center element 203 can detect the acoustic wave from around the center 182 of curvature of the casing 184 with high sensitivity since the normal line of each element 201 surrounding the center element 203 do not pass the center 182 of curvature of the casing 184. Accordingly, the resolution distribution in a region of an inspection target can be improved, and an image quality can be raised. According to a size of the region of the inspection target, the elements 201 can be arranged in a desired form. For example, in a case where a distance between a center of the center element 203 and a center of the element 201 surrounding the center element 203 is 2.1 mm, a signal from a region within a radius of 2.1 mm from the center 182 of curvature of the casing 184 can be detected with high sensitivity. All of the ultrasonic transducers 200 mounted to the casing 184 can be an ultrasonic transducer 200 illustrated in FIG. 2A. Alternatively, ultrasonic transducers 200 having different arrangements of the elements 201 illustrated in FIGS. 2A to 2C can be arranged. In comparison with the above-described arrangement, in a case where all the normal lines of the seven (7) ultrasonic transducer elements 201 pass through the center 182 of curvature of the casing 184, the acoustic wave from around the center 182 of curvature cannot be detected with high sensitivity. Hence, the resolution distribution in the region of the inspection target appears, and the image quality is likely to be degraded.

Further, when the subject 120 is locally present in the center 182 of curvature of the casing 184, sensitivity characteristics of the ultrasonic transducer elements 201 surrounding the center element 203 in the ultrasonic transducer 200 having seven (7) ultrasonic transducer elements 201 as illustrated in FIG. 2A can be different from sensitivity characteristics of the center element 203. The sensitivity characteristics of the elements 201 surrounding the center element 203 are desirably made higher than the sensitivity characteristics of the center element 203. This is because if the same elements as the center element 203 are used for the ultrasonic transducer elements 201 surrounding the center element 203, the detection sensitivity is lowered due to the directionality of the elements 201 not facing the center 182 of curvature.

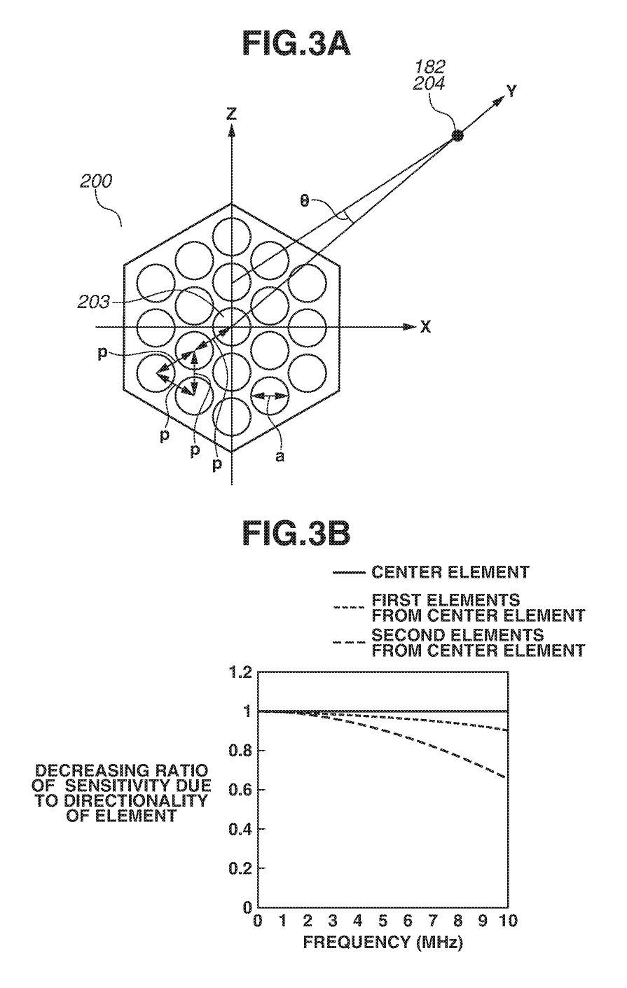

With reference to FIGS. 3A and 3B, description will be given on decrease in the detection sensitivity due to the directionality of the ultrasonic transducer element 201 in a case, as illustrated in FIG. 2C, where the element 201 is disposed at a position L mm away from the center element 203 facing the center 182 of curvature. The directionality of element in a case of a circular flat plate vibrator and directionality of the element in a case of a rectangular vibrator can be respectively written by the following formulae.

R(θ)_{circle}=|2J₁(k*a*sin θ)/(k*a*sin θ)|

R(θ)_{square}=|sin(k*a*sin θ)/(k*a*sin θ)|

J₁ is the Bessel function of the first type, k is a wavenumber (k=2*π/λ) calculated from a ratio n of a circumference of a circle to its diameter, and a wavelength λ of the ultrasonic wave. In a case of the circular flat plate vibrator, a is a diameter of the vibrator. In a case of the rectangular vibrator, a is a length of a side. Θ is an angle formed between the normal line connecting the center element 203 to the center 182 of curvature of the casing 184, and a line connecting a center of each element 201 to the center 182 of curvature of the casing 184.

The diameter a of the ultrasonic transducer element 201 is 2 mm, and an interval p between the elements is 2.1 mm. A point sound source 204 is disposed at the center 182 of curvature of the casing 184 as illustrated in FIG. 3A, and the detection sensitivity of the center element 203 is made equal to 1 when a distance between the center element 203 and the point sound source 204 is 100 mm. In such a case, the directionality of the element 201 surrounding the center element 203 shows the directionality as illustrated in FIG. 3B. As illustrated in FIG. 3B, due to the directionality of the element 201, the detection sensitivity of first elements from the center element 203 decreases about 10% at 10 MHz. Further, the detection sensitivity of second elements from the center element 203 decreases about 24% at 10 MHz. Thus, the decrease in the detection sensitivity due to the directionality is larger on a side of low frequency than on a side of high frequency. Therefore, it is desirable to make, particularly on the side of high frequency, the sensitivity characteristics of the elements 201 arranged around the center element 203 higher than the sensitivity characteristics of the center element 203. This is because the sensitivity and resolution of the acoustic wave sensor can be improved. Further, it is desirable to increase the sensitivity characteristics of the elements 201 arranged around the center element 203 on the side of high frequency as the element 201 arranged around the center element 203 are arranged farther apart from the center element 203, according to the number of the elements 201. Thus, the detection sensitivity on the side of high frequency can be increased. The high frequency means a frequency higher than a frequency in a peak sensitivity of the center element 203.

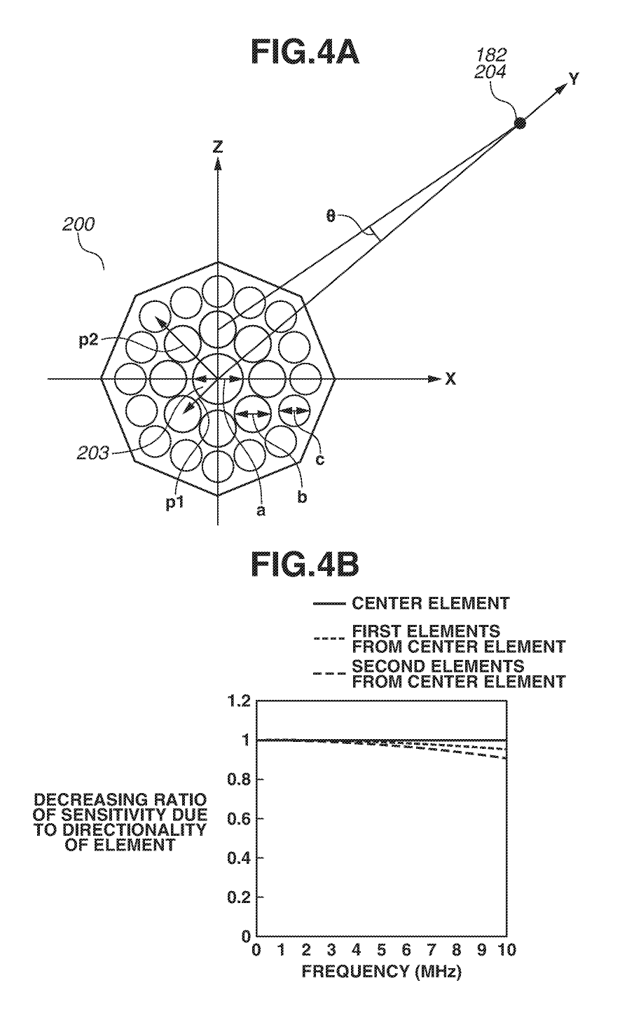

The directionality of the element increases in proportion to a size of the element. Accordingly, the directionality of the element decreases as the size of the element decreases. When a diameter of the element 201 arranged around the center element 203 is made smaller than a diameter of the center element 203 as illustrate in FIG. 4A (i.e., a diameter b=1.5 mm, and a diameter c=1.2 mm), it is possible to decrease the directionality of the second elements from the center element 203 about 10% at 10 MHz. Here, p1=2.1 mm, and p2=4.2 mm. Thus, when the size of the element 201 arranged around the center element 203 is made smaller than the size of the center element 203 to increase the detection sensitivity, the sensitivity and resolution of the acoustic wave sensor can be desirably improved. Further, when the sizes of the elements 201 arranged around the center element 203 are made smaller as the elements 201 arranged around the center element 203 are arranged farther apart from the center element 203, according the number of the elements 201, the detection sensitivity can be desirably increased.

The ultrasonic transducers 200 are arranged on the concave surface of the casing 184 of the ultrasonic unit 180 as illustrated in FIG. 1B. In the example illustrated in FIG. 1B, the ultrasonic transducers 200 are disposed along a generating line extending from an upper edge of the concave surface of the casing to a bottom center of the casing at an approximately equal interval, and this arrangement is repeated about a center of the casing 184 at an approximately equi-angular interval. Accordingly, it is possible to arrange the ultrasonic transducer elements 201 so as to face the center 182 of curvature of the casing 184 with a comparatively high density.

### <Processing Portion>

Returning to FIG. 1, the processing portion 160 and the image display portion 170 will be described. The photoacoustic wave is detected by the ultrasonic transducer (or sensor) 200 disposed in the ultrasonic unit 180. The photoacoustic wave generated in the subject 120 is transmitted in approximately (360 degrees)*(180 degrees) directions, and detected. Regarding an image reconstruction, it is possible to use back projection or the like in Time Domain or Fourier Domain Optical Coherence Tomography usually used in the tomography technology. The Fourier Domain Optical Coherence Tomography is often used to acquire a 3D image with high resolution, but a method of the image reconstruction is not limited to this method. The intensity of the photoacoustic wave emitted from a region of the subject 120 is calculated in the processing portion 160. To increase a rate of image forming processing, the processing portion 160 calculates a value determined from a position of the ultrasonic transducer 200, a position of a region of the subject 120, and a receiving time, as a function of a radius of a sphere of the ultrasonic unit 180, and stores its factor in a memory. A received signal of each ultrasonic transducer 200 is multiplied by the factor, and the multiplication result is accumulated for each region. Thus, image data can be formed. In such a manner, the image data on the spherical surface is calculated, and image processing of the image information is executed using the Fourier Domain Optical Coherence Tomography. Accordingly, the 3D image of the subject 120 can be displayed on the image display portion 170 with high resolution.

According to the above-described exemplary embodiment, a variety of elements including the ultrasonic transducer elements facing approximately the center of curvature and the ultrasonic transducer elements facing the points off from the center of curvature are arranged on the concave portion of the probe casing. Therefore, the resolution distribution in a region of the subject can be improved, and the image quality can be improved. In a case where the ultrasonic transducer elements are mounted to the probe casing one by one, it is likely to cause increase in cost due to increase in the number of mounting processes, and increase in weight and size of the ultrasonic transducer unit. However, in the ultrasonic transducer unit according to the present exemplary embodiment, a substrate to which a plurality of the ultrasonic transducer elements is disposed is mounted to the hemispherical inner surface, so that reduction in cost, weight, and size can be attained. It is because that a plurality of the ultrasonic transducer elements can be collectively provided on the substrate by using the semiconductor process, and the reduction in cost can be achieved. Further, in a case where each element is mounted to the probe casing, it is necessary to individually mount an electric circuit, such as a detection circuit and a signal amplification circuit, and the like. Hence, it is likely to increase weight and size. However, when an electric circuit is provided for each substrate to which a plurality of the elements is disposed, it is possible to suppress the increase in weight and size.

Hereinafter, aspects of the present invention will be described with more specific exemplary embodiments.

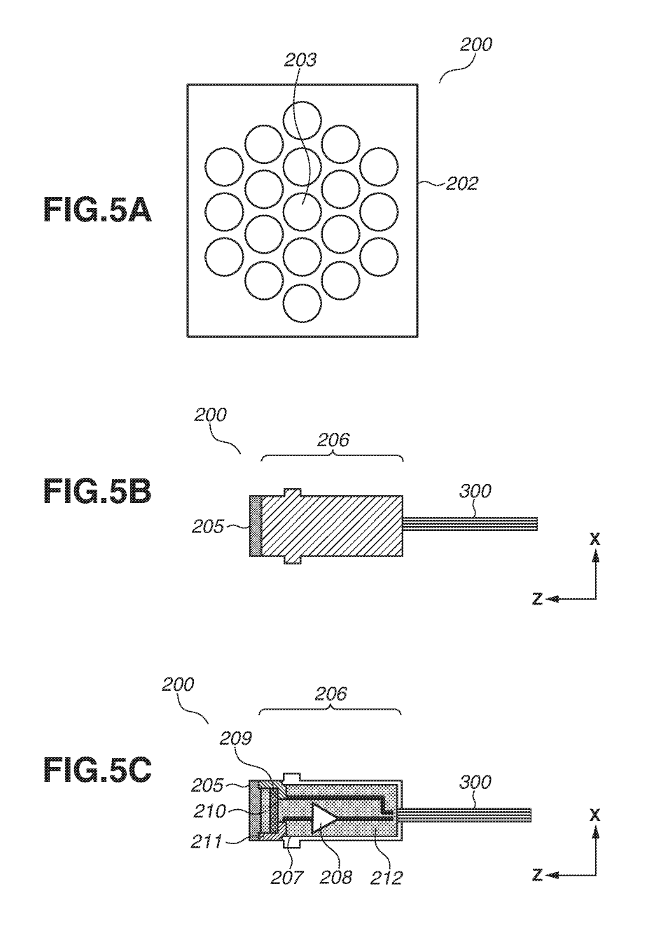

Specific examples of the ultrasonic unit or photoacoustic diagnosis apparatus according to the present exemplary embodiment will be described. In a first exemplary embodiment, a near-infrared nanopulse laser is used as a light source 150. Here, a titanium sapphire laser is used, and an Nd:YAG laser is used as an excitation light source. When the subject is irradiated with light at a wavelength near 800 nm, the photoacoustic wave is generated in the subject 120. A phantom for a breast is used as the subject 120. A film of polymethylpentene is used as a shape retaining unit 110. As for an acoustic matching material 130, water is used to fill a space between the shape retaining unit 110 and an ultrasonic unit 180. In the ultrasonic unit 180, an inside radius of the approximately hemispherical surface is set to approximately 120 mm, and 150 ultrasonic transducers 200 are arranged in a manner such that the ultrasonic wave receiving surfaces face approximately the center of curvature of the hemispherical surface. The ultrasonic transducer 200 includes 19 ultrasonic transducer elements 201 as illustrated in FIG. 5A. The ultrasonic transducer element 201 is a capacitive transducer, and a casing 184 of the ultrasonic unit 180 is made of aluminum.

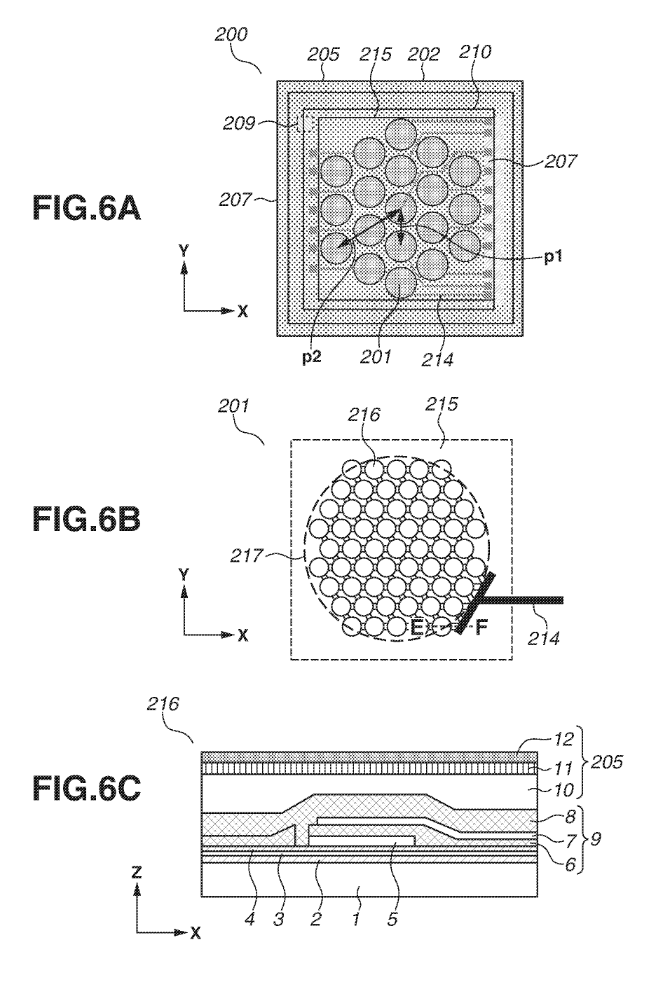

The ultrasonic transducer 200 will be described referring to FIGS. 5B and 5C, and FIG. 6. FIG. 5B is a view illustrating an example of the ultrasonic transducer, and FIG. 5C is a cross-sectional view taken along an X-Z plane illustrated in FIG. 5B. The ultrasonic transducer 200 includes a protection layer 205, a body 206, and a wiring line 300, and a protrusion is formed in a portion of the body 206. In the body 206, there are arranged an element group 210 of the elements 201 (capacitive transducer type), a second flexible wiring line 207, a first flexible wiring line 209, and a receiving preamplifier 208 supported by a wiring line substrate 212. Thus, the ultrasonic transducer 200 includes the element group 210 disposed on the sensor plane. The element group 210 executes at least one of conversion of the acoustic wave into a received signal and conversion of a transmission signal into the acoustic wave. The element group 210 is supported by a support member 211.

FIG. 6A is a schematic view illustrating the sensor plane of the ultrasonic transducer 200. A protection layer 205 is formed on an outermost surface of the sensor plane. An external configuration 202 of the ultrasonic transducer 200 is quadrangular, and the element group 210 fabricated on a silicon substrate is disposed in the external configuration 202. The element group 210 includes 19 elements 201. In each element 201, an electrode 214 (a second electrode 7 described below) is drawn out to an edge of the silicon substrate, connected to the second flexible wiring line 207, and connected to the receiving preamplifier 208 in the body 206. An electrode 215 (a first electrode 3 described below) which is the other one of the electrode 214 is connected to the first flexible wiring line 209 as a common electrode.

With reference to FIGS. 6B and 6C, the element 201 will be further described. FIG. 6B is an enlarged schematic view illustrating a side of the sensor plane of the element 201. FIG. 6C is a cross-sectional view taken along an E-F plane illustrated in FIG. 6B. The ultrasonic transducer element 201 is an assembly of a plurality of cells 216. The cell 216 includes a silicon substrate 1, a first insulation layer 2, the first electrode 3, a second insulation layer 4 formed on the first electrode 3, and a vibration film 9. The vibration film 9 includes a third insulation layer 6 formed on the second insulation layer 4 with a cavity 5 interposed therebetween, a second electrode 7 on the insulation layer 6, a fourth insulation layer 8 on the electrode 7. On the vibration film 9, a support layer 11 is bonded through an adhesive layer 10. A light reflection layer 12 is formed on the support layer 11. Thus, a protection layer 205 is formed. The element 201 includes a plurality of the thus-configured cells 216. In the example illustrated in FIG. 6B, the element 201 includes 59 cells 216, but the element 201 can include one cell, or a large number of cells. Further, an arrangement pattern of the cells 216 can be any of a square-block pattern, a staggered pattern, and a honeycomb-shaped pattern (a hexagonal closest packing structure with a small space between cells). The shape of the cell can be any of a circular shape, a rectangular shape, a square shape, a polygonal shape, and the like. Further, the external configuration of the element 201 illustrated in FIG. 6B is an approximately circular shape 217, but it can be circular, polygonal, or the like. In the example illustrated in FIG. 6A, the first electrode 3 and the second electrode 7 are drawn out from a side of the receiving surface through the flexible wiring line. However, it is possible to form a through hole in the silicon substrate 1, form an electrode directly on a backside of the silicon substrate 1, and connect the electrode to the circuit substrate.

The protection layer 205 including the light reflection layer 12 is formed on the surface of the element 201. The light reflection layer 12 serves to reduce the photoacoustic wave generated due to irradiation of the element with scattering light of the pulse light from the light source 150 and with the reflection light from the shape retaining unit 110. The light reflection layer 12 is a layer made by an Au deposition, and a PET film with a thickness of 12 μm is used as the support layer 11 on which the Au deposition is executed. An adhesive of a silicon type is used as the adhesive layer 10 to fabricate the protection layer 205. The type and thickness of the light reflection layer 12, the support layer 11, and the adhesive layer 10 are not limited to those described above.

In the element 201, a bias voltage can be applied to the first electrode 3 by a voltage applying unit. When the bias voltage is applied to the first electrode 3, a potential difference appears between the first electrode 3 and the second electrode 7. Due to the potential difference, the vibration film 9 is displaced to a position where a restoration force of the vibration film 9 counterbalances an electrostatic attractive force. In this state, when the ultrasonic wave reaches the vibration film 9, the vibration film 9 is vibrated. Hence, an electrostatic capacitance between the first electrode 3 and the second electrode 7 is changed, and a current flows in the second electrode 7. The current can be taken out as an electrical signal of the ultrasonic wave. At the time of receiving the signal, a portion for controlling bias voltage applies a receiving bias voltage according to instructions from a control unit (not illustrated). The ultrasonic wave, such as the photoacoustic wave, generated in the subject 120 is received by the ultrasonic wave sensor, and a received signal is acquired. The received signal is amplified by the receiving preamplifier 208, and the amplified signal is supplied to the processing portion 160.

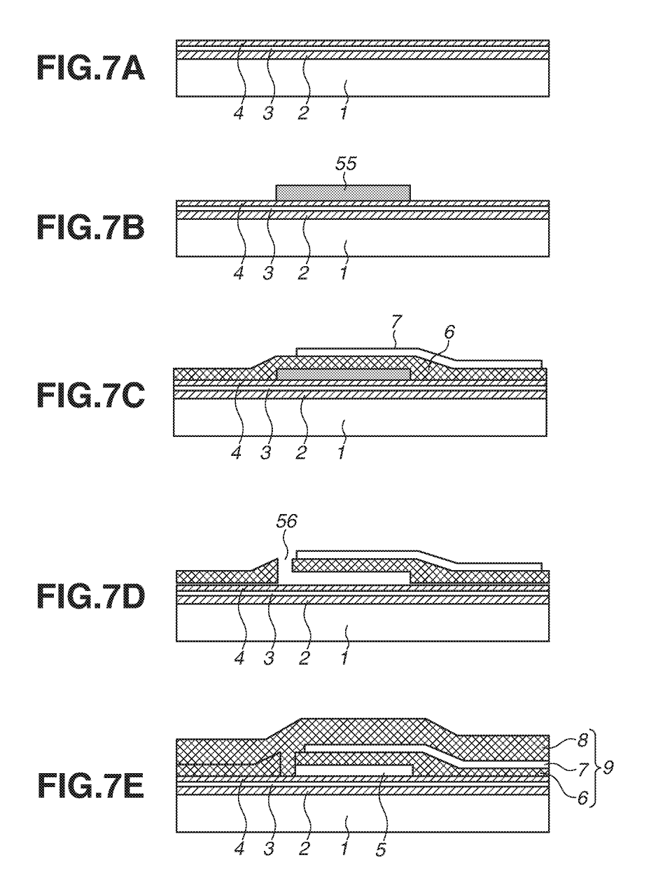

According to FIGS. 7A to 7E, an example of a fabrication method of the element 201 (capacitive transducer type) will be described. As illustrated in FIG. 7A, a first insulation layer 2 is formed on a substrate 1. The substrate 1 is a silicon substrate, and the first insulation layer 2 serves to insulate the substrate 1 from a first electrode 3. Then, the first electrode 3 is formed. The first electrode 3 is desirably made of a conductive material with a small surface roughness, such as titanium, tungsten, and aluminum. In a case where the surface roughness of the first electrode 3 is large, a distance between the first electrode 3 and a second electrode 7 varies among the elements due to the surface roughness. Therefore, the conductive material with a small surface roughness is desirable. Then, a second insulation layer 4 is formed. The second insulation layer 4 is desirably an insulation material with a small surface roughness, too. The second insulation layer 4 is formed to prevent an electrical short circuit or insulation breakdown, between the first electrode 3 and the second electrode 7, which possibly appears when the voltage is applied between the first electrode 3 and the second electrode 7. Further, the second insulation layer 4 is formed to prevent the first electrode 3 from being etched when a sacrifice layer 55 is removed in the following step to be executed after the present step. The second insulation layer 4 is made of material, such as silicon nitride or silicon oxide.

Next, as illustrated in FIG. 7B, the sacrifice layer 55 is formed. The sacrifice layer 55 is changed to a cavity 5. The sacrifice layer 55 is desirably made of material with a small surface roughness. In a case where the surface roughness of the sacrifice layer 55 is large, a distance between the first electrode 3 and the second electrode 7 varies among the elements. Therefore, the sacrifice layer 55 with a small surface roughness is desirable. Further, in order to shorten an etching time during which the sacrifice layer is removed, it is desirable to use material whose etching rate is high. The sacrifice layer 55 is made of material, such as amorphous silicon, polyimide, and chromium. An etching liquid for chromium almost never etches a film of silicon nitride or silicon oxide. Therefore, chromium is desirable when the insulation layer 4 and a vibration film (described below) are made of silicon nitride or silicon oxide.

Next, as illustrated in FIG. 7C, a third insulation layer 6 is formed. The third insulation layer 6 preferably has a low tensile stress, for example, a tensile stress not more than 500 MPa. A stress of the silicon nitride film can be controlled, and the low tensile stress can be adjusted to a value not more than 500 MPa. In a case where the vibration film has a compressive stress, it is likely that sticking or buckling of the vibration film occurs. Hence, the vibration film is likely to be largely deformed. On the other hand, in a case where the vibration film has a large tensile stress, the third insulation layer 6 is likely to be broken. Accordingly, it is desirable that the third insulation layer 6 has a low tensile stress. For example, the third insulation layer 6 is desirably a silicon nitride film of which stress can be controlled and in which a tensile stress can be adjusted to low. Then, the second electrode 7 is formed. The second electrode 7 is desirably made of material of which residual stress is small. Therefore, the second electrode 7 is desirably made of a metal, such as aluminum, alloy of aluminum and silicon, and titanium. However, the material of the second electrode 7 is not limited to those materials.

Next, as illustrated in FIG. 7D, an etching hole 56 is formed in the third insulation layer 6. The etching hole 56 is a hole through which an etching liquid or an etching gas is introduced to etch the sacrifice layer 55. Then, the sacrifice layer 55 is removed to form the cavity 5. As a method of removing the sacrifice layer 55, wet etching and dry etching are desirable. When chromium is used as material of the sacrifice layer 55, the wet etching is desirable.

Next, as illustrated in FIG. 7E, a fourth insulation layer 8 is formed to seal the etching hole 56. The vibration film 9 is formed of the third insulation layer 6, the second electrode 7, and the fourth insulation layer 8. As the sealing material, the same material as the third insulation layer 6 is desirable since this material has adhesion property. When the third insulation layer 6 is made of silicon nitride, it is desirable that the fourth insulation layer 8 is also made of silicon nitride.

In FIGS. 6C and 7A to 7E, there is illustrated an example in which the second electrode 7 is interposed between the third insulation layer 6 and the fourth insulation layer 8. However, the following method can be also adopted. After the third insulation layer 6 is formed, the etching hole 56 is formed by the etching of the sacrifice layer. Then, after the fourth insulation layer 8 is formed, the second electrode 7 is formed. However, in such a case, a possibility of a short circuit of the element due to a foreign substance or the like is likely to increase when an outermost surface of the second electrode 7 is exposed. Accordingly, the second electrode 7 is preferably interposed between the insulation layers. In the present exemplary embodiment, to prepare the element group 210, the elements 201 are arranged in a manner illustrated in FIG. 6A, and the silicon substrate is cut to a size of the element group 210. Through the above-described steps, it is possible to fabricate the element 201 and the cell 216 as illustrated in FIGS. 6B and 6C, and the element group 210 as illustrated in FIG. 6A.

The element group 210 is fixed to a support member 211 illustrated in FIG. 5C with resin adhesive, such as epoxy resin, and a first electrode 215 and a second electrode 214 are connected to a first flexible wiring line 209 and a second flexible wiring line 207, respectively. The flexible wiring lines 207 and 209 are connected to a wiring line substrate 212 in which a receiving amplifier 208 is disposed. A collective set of the element group 210, the flexible wiring lines 207 and 209, and the wiring line substrate 212 is stored in the body 206. The body 206 can be formed by using resin, and the like. After the collective set is stored in the body 206, the body 206 is sealed by an adhesive such that no infiltration of the acoustic matching material, and the like into the body 206 occurs. On a surface of the element group 210 stored in the body 206, the protection layer 205 including the light reflection layer 12 is formed. The light reflection layer 12 is made of Au. As the support layer 11, a PET film with a thickness of 12 μm is used. After Au is deposited on the support layer 11, the support layer 11 having the deposited Au is bonded on a surface of the element group 210 using an adhesive of a silicon type. Then, an unnecessary portion of the support layer 11 is cut, and the protection layer 205 of the element group 210 is fabricated.

Through the above steps, the ultrasonic transducer 200 as illustrated in FIGS. 5B and 5C can be fabricated. In this configuration of each element 201, the first electrode 215 is connected to the first flexible wiring line 209 in common, and the first flexible wiring line 209 is connected to a power source for applying a bias voltage through the wiring line 300. The second electrode 214 is connected to the receiving amplifier 208 through the second flexible wiring line 207, and the second flexible wiring line 207 is connected to the processing portion 160 through the wiring line 300.

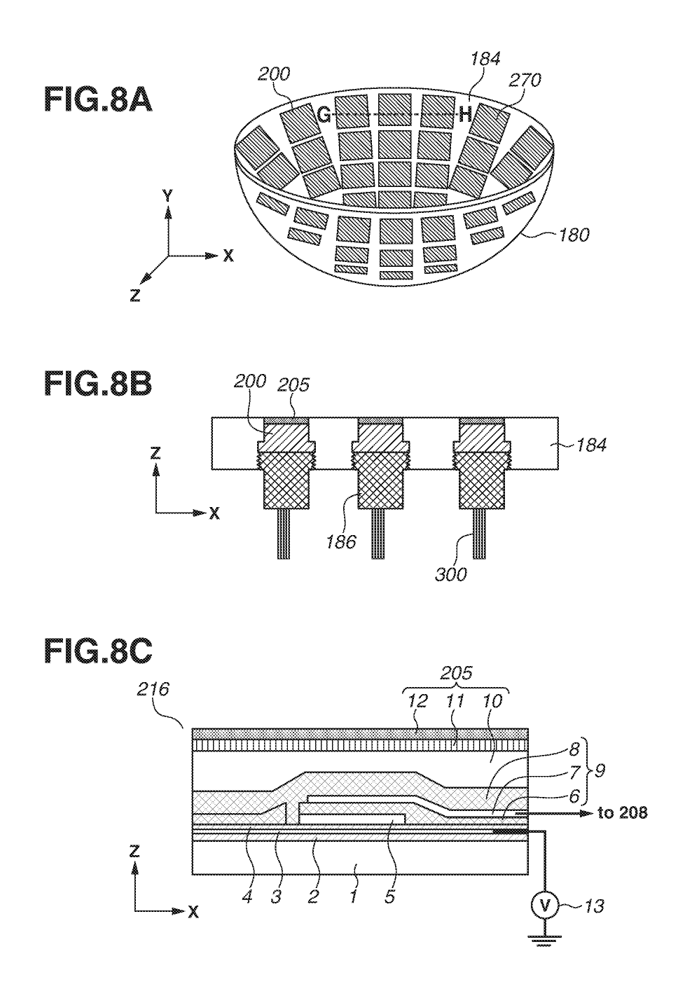

The ultrasonic transducer 200 fabricated as described above is disposed in the casing 184 with a hole 270 as illustrated in FIG. 8A. In a manner illustrated in FIG. 8B, a positional alignment is carried out using a concave and convex on a wall surface of the hole 270 and a concave and convex of the ultrasonic transducer 200 illustrated in FIG. 5B. The ultrasonic transducer 200 is screwed and clamped from a side of the wiring line 300 of the ultrasonic transducer 200 using a fixture 186. Since an inner space of the ultrasonic unit 180 is filled with a medium at the time of measurement, it is desirable that an O-ring of silicon rubber, or the like is disposed in a space between the casing 184 and the ultrasonic transducer 200. Alternatively, it is desirable that the space is filled up with an adhesive. Thus, the ultrasonic unit 180 as illustrated in FIG. 1A can be fabricated.

Regarding the above-described element group 210 in which all of the elements 201 have the same characteristics, specific configuration and characteristics will be described. The element 201 has, as illustrated in FIG. 6B, an approximately circular shape 217 having a diameter of 2 mm. The element group 210 includes 19 elements 201 as illustrated in FIG. 6A. Intervals p1 and p2 of the elements 201 are 2.1 mm and 4.2 mm, respectively. The cell 216 has a circular shape, and a diameter of the cavity 5 is 36 μm. An interval between cells 216 adjacent to each other is 39 μm. In FIG. 6B, the cells 216 are illustrated in an abbreviated manner, and the actual overall number of the cells 216 arranged in the element 201 is 2400.

As illustrated in FIG. 8C, the cell 216 includes a silicon substrate 1 having a thickness of 300 μm, a first insulation layer 2 having a thickness of 1000 nm and formed on the silicon substrate 1, a first electrode 3 having a thickness of 100 nm and formed on the first insulation layer 2, and a second insulation layer 4 having a thickness of 350 nm and formed on the first electrode 3. Further, the cell 216 includes a vibration film 9. The vibration film 9 is supported in a vibratory manner, and includes a second electrode 7 having a thickness of 100 nm, a third insulation layer 6 having a thickness of 400 nm, and a fourth insulation layer 8 having a thickness of 450 nm, and a cavity 5. A height of the cavity 5 is 140 nm. The first electrode 3 serving as a common electrode is connected to a first flexible wiring line 209, and connected through a wiring line 300 to a voltage applying unit which applies a bias voltage between the first electrode 3 and the second electrode 7. The second electrode 7 is connected to a receiving preamplifier 208 through a second flexible wiring line 207. The ultrasonic transducer 200 is fabricated using those elements 201 illustrated in FIGS. 5B and 5C, and FIG. 6A. The ultrasonic unit 180 can be fabricated by assembling those ultrasonic transducers 200 in a manner illustrated in FIGS. 8A and 8B.

Characteristics of the element 201 according to the present exemplary embodiment will be described. When the element 201 receives the ultrasonic wave, a voltage applying unit 13 applies a direct current (DC) voltage to the first electrode 3 to generate a potential difference between the first electrode 3 and the second electrode 7. Upon receipt of the ultrasonic wave, the vibration film 9 having the second electrode 7 is deformed. Hence, the interval between the second electrode 7 and the first electrode 3 (a distance in a height direction of the cavity 5) is changed to change an electrostatic capacitance. A current flows in the second electrode 7 according to the change in the electrostatic capacitance. The current output from the cell 216 is amplified, and converted into a voltage by the receiving preamplifier 208. Thus, the ultrasonic wave is taken out as an electrical signal.

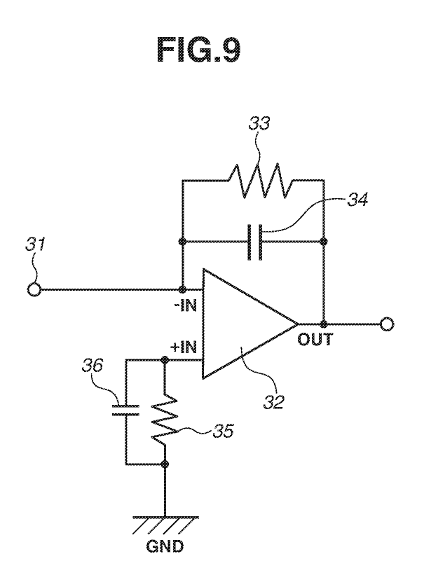

FIG. 9 is a diagram illustrating a configuration example of the receiving preamplifier 208 according to the present exemplary embodiment. The receiving preamplifier 208 is a transimpedance circuit. The transimpedance circuit includes an operational amplifier 32, feedback resistors 33 and 35, and feedback capacitors 34 and 36. The operational amplifier 32 is connected to positive and negative power sources (VDD and VSS), and an inverting input terminal (−IN) is connected to the second electrode 7 of the element 201. An output terminal (OUT) is connected to the inverting input terminal (−IN) through the feedback resistor 33 and the feedback capacitor 34 which are connected in parallel. Thus, an output signal is fed back. A non-inverting input terminal (+IN) is connected to a ground terminal (GND) through the feedback resistor 35 and the feedback capacitor 36 which are connected in parallel. A voltage of the ground terminal is set to an intermediate potential between the positive power source VDD and the negative power source VSS. Resistance values of the feedback resistors 33 and 35 are equal to each other, and capacitance values of the feedback capacitors 34 and 36 are equal to each other.

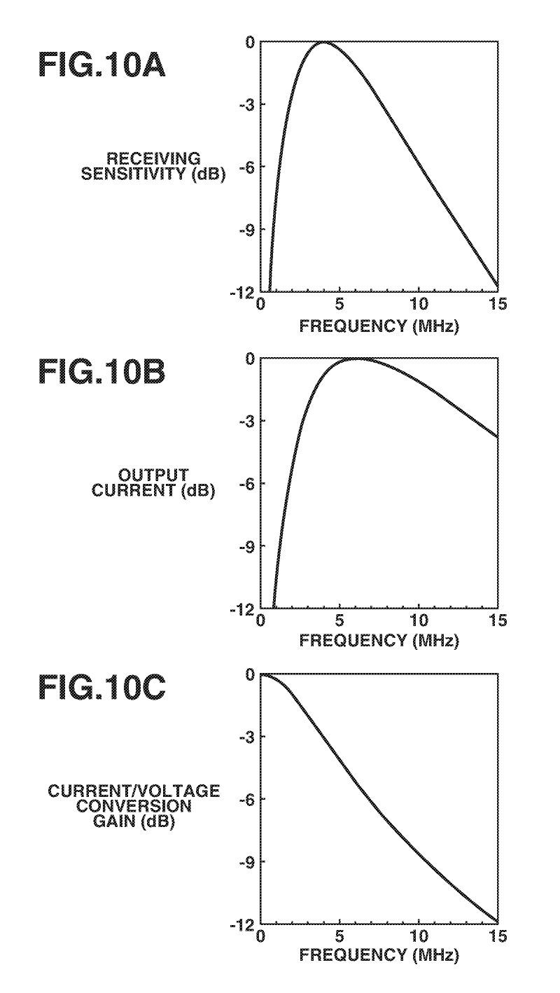

FIG. 10A is a graph illustrating frequency characteristics of a receiving sensitivity of the element 201 according to the present exemplary embodiment. The frequency characteristics are frequency characteristics of a voltage signal which is obtained by amplification and conversion of the output current generated from the received ultrasonic wave in the ultrasonic transducer 200. The amplification and conversion are executed by the receiving preamplifier 208. A value on an axis of ordinates is normalized by a peak value of the receiving sensitivity. FIG. 10B is a graph illustrating characteristics of an output current of the element 201, and FIG. 10C is a graph illustrating gain characteristics of the receiving preamplifier 208. A receiving band of the element 201 illustrated in FIG. 10A is determined by a product of the output current characteristics of the element 201 and the gain characteristics of the receiving preamplifier 208.

An output current I of the CMUT can be written by the following formulae 1 and 2 when a change in the electrostatic capacitance in a parallel flat plate structure approximates a change in the electrostatic capacitance in the CMUT.

I=P/((Zm+Zr)/(∈S*Vb/d²)+j ωC) (1)

Zm=j*km*(ω/ω₀²)−1/ω) (2)

Here, P is a pressure of the acoustic wave, ∈ is a dielectric constant of vacuum, S is an area of the second electrode, Vb is a bias voltage applied between two electrodes, d is a gap between the electrodes, Zm is a mechanical impedance of the vibration film, and Zr is an acoustic impedance of the medium. Further, ω is an angular frequency of the acoustic wave, C is an overall electrostatic capacitance, km is a spring constant of the vibration film, and ω₀ is a resonance frequency. In the formula 1, since the overall electrostatic capacitance C is relatively small, a function of the frequency is the mechanical impedance Zm of the vibration film. Further, the CMUT is normally used with its surface in contact with liquid or gel. The acoustic impedance Zr of the liquid is larger than the mechanical impedance Zm of the vibration film. Accordingly, the acoustic impedance Zr of the liquid largely influences the frequency characteristics of the output current as illustrated in FIG. 10B. The resonance frequency of the vibration film is a frequency at which the mechanical impedance Zm of the vibration film is zero (0). At this frequency, the output current becomes the highest as illustrated in FIG. 10B. In FIG. 10B, the peak frequency of the output current is 6 MHz.

Gain characteristics and a cutoff frequency, illustrated in FIG. 10C, of the detection circuit can be written by formulae 3 and 4, respectively.

G=Rf/(1+jωRf*Cf) (3)

F≈1/(2πRf*Cf) (4)

Here, G is a circuit gain, Rf is the feedback resistance value, Cf is the feedback capacitance value, c is an angular frequency of the input current, and f is the cutoff frequency.

Further, in order for stable driving of the circuit illustrated in FIG. 9, it is necessary to satisfy formula 5.

Cf≧((Cin)/(π*GBW*Rf))^{0.5} (5)

Here, GBW is Gain Bandwidth Product (an amplifier gain 0 dB(=1)*frequency) of the operational amplifier, Cin is a parasitic capacitance in the inverting input terminal (−IN) of the operational amplifier. Generally, when Cin is large, operation of the operational amplifier exceeds its capacity, and the negative feedback circuit becomes unstable. Hence, the circuit itself oscillates, and the current/voltage conversion cannot be carried out. Therefore, it is necessary to select appropriate GBW, Rf, and Cf for a value of Cin.

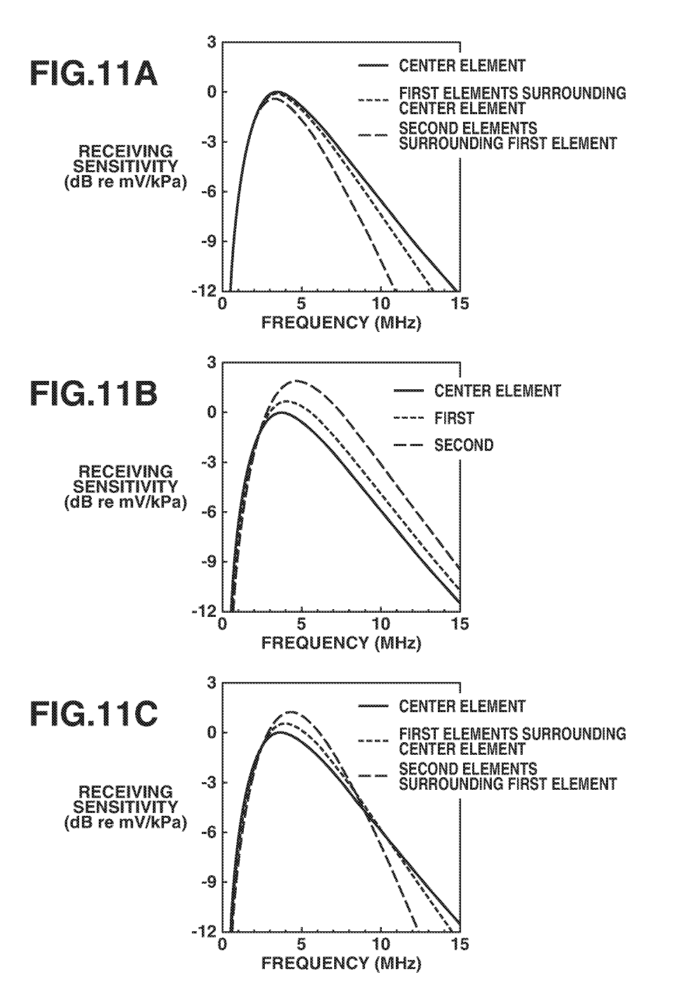

In the present exemplary embodiment, the feedback resistance value and the feedback capacitance value of the receiving preamplifier 208 is set to 3480 Ω and 15 pF, respectively. A capacitance value of the element 201 is 125 pF. Each of the 19 elements 201 in the element group 210 is connected to a different receiving preamplifier 208. Among the receiving preamplifiers 208, a value of the feedback capacitance value is equal to each other, and the feedback resistance value is equal to each other. FIG. 11A is a graph illustrating the frequency characteristics of the receiving sensitivity of the element group 210. The frequency characteristics illustrated in FIG. 11A is frequency characteristics of a voltage signal which is obtained by amplification and conversion of the output current generated from the received ultrasonic wave in the ultrasonic transducer 200. The amplification and conversion are executed by the receiving preamplifier 208. At this time, a distance between a center of the ultrasonic transducer 200 and a point sound source 204 is 100 mm, and a spherical wave is generated by the point sound source 204. A value on an axis of ordinates is normalized by a peak value of the receiving sensitivity of the ultrasonic transducer element 201 disposed at a center.

In a case where a size of the subject 120 has a radius of 4.2 mm with its center being at the center 182 of curvature, the acoustic wave from the center 182 of curvature has sensitivity characteristics designated by “center element” in FIG. 11A. Further, sensitivity characteristics of the acoustic waves from positions, which are respectively 2.1 mm and 4.2 mm away from the center 182 of curvature, have also sensitivity characteristics designated by “center element”. A reason therefore is that the ultrasonic transducer element 201 surrounding the center element 201 can detect the acoustic wave generated from around the center 182 of curvature of the casing 184 with high sensitivity since normal lines of the ultrasonic transducer elements 201 surrounding the center element 201 do not pass the center 182 of curvature. Hence, signals from an entire subject 120 can be detected with high sensitivity. When the ultrasonic transducer elements 201 are appropriately arranged according to the size of the subject 120, the acoustic wave generated around the center 182 of curvature can be detected with high sensitivity.

A second exemplary embodiment will be described. In the present exemplary embodiment, a circuit constant is changed to increase the sensitivity. A case where a size of the subject 120 is approximately equal to the center 182 of curvature will be described. When the acoustic wave generated at the center 182 of curvature is received by the ultrasonic transducer 200 which is an element group as illustrated in FIG. 6A, the characteristics of the receiving sensitivity become those as illustrated in FIG. 11A. In FIG. 11A, “center element” indicates characteristics of the ultrasonic transducer element 201 disposed at a center (center element), and “first elements surrounding center element” indicates characteristics of the ultrasonic transducer elements 201 disposed around the center element (first ultrasonic transducer elements). Further, “second elements surrounding first elements” indicates characteristics of the ultrasonic transducer elements 201 disposed around the first ultrasonic transducer elements surrounding the center element (second ultrasonic transducer elements). The output current characteristics and the current/voltage conversion gain of the ultrasonic transducer elements 201 in the ultrasonic transducer 200 are equal to each other. Therefore, due to the directionality of the ultrasonic transducer element 201, the receiving sensitivities of the first and second ultrasonic transducer elements 201 decrease. When the receiving sensitivities of the center, first, and second ultrasonic transducer elements at 10 MHz are compared with each other, the receiving sensitivity of the first ultrasonic transducer elements surrounding the center element is approximately 10% lower than the receiving sensitivity of the center element. The receiving sensitivity of the second ultrasonic transducer elements 201 surrounding the first ultrasonic transducer elements 201 is approximately 24% lower than the receiving sensitivity of the center element. In such a case, it is desirable that the feedback resistance value and the feedback capacitance value of the receiving preamplifier 208 are changed to increase the detection sensitivity to the acoustic wave generated at the center 182 of curvature.

There are prepared three types of combinations of the feedback resistance value and the feedback capacitance value of the receiving preamplifier 208 to be connected to the ultrasonic transducer element 201. In the first combination, the feedback resistance value is 3480Ω, and the feedback capacitance value is 15 pF. In the second combination, the feedback resistance value is 3240Ω, and the feedback capacitance value is 13 pF. In the third combination, the feedback resistance value is 2940Ω, and the feedback capacitance value is 10 pF. The parameters and configurations other than the receiving preamplifier 208 are the same as those in the first exemplary embodiment. The ultrasonic transducer 200 and the ultrasonic unit 180 can be fabricated by the same method as in the first exemplary embodiment. FIG. 11B is a graph illustrating the frequency characteristics of the receiving sensitivity in the cases of where three types of the receiving preamplifiers 208 are respectively connected to the elements 201. The output current characteristics of the elements 201 are the same with each other. Further, a distance between a center of the element 201 and a point sound source 204 is 100 mm, and a spherical wave is generated at the point sound source 204. The characteristics are frequency characteristics of a voltage signal which is obtained by amplification and conversion of the output current generated from the received ultrasonic wave. The amplification and conversion are executed by the receiving preamplifier 208. A value on an axis of ordinates is normalized by a peak value of the receiving sensitivity of the element 201 connected to the receiving preamplifier 208 of the first combination.

In FIG. 11B, “center element” indicates the receiving sensitivity characteristics at the time of when the elements 201 are connected to the same receiving preamplifier as in the first exemplary embodiment, and “first” indicates the receiving sensitivity characteristics at the time of when the elements 201 are connected to the receiving preamplifier of the second combination. Further, “second” indicates the receiving sensitivity characteristics at the time of when the elements 201 are connected to the receiving preamplifier of the third combination. By changing the current/voltage conversion gain of the receiving preamplifier 208, the receiving sensitivity on a side of high frequency of the first ultrasonic transducer elements is made larger than that of the center element, and the receiving sensitivity on a side of high frequency of the second ultrasonic transducer elements is made larger than that of the first ultrasonic transducer elements. At the center illustrated in FIG. 6A, the element 201 with such receiving sensitivity characteristics connected to the first receiving preamplifier is arranged. For the first ultrasonic transducer elements surrounding center element, the element 201 connected to the second receiving preamplifier is arranged. For the second ultrasonic transducer elements surrounding the first ultrasonic transducer elements, the element 201 connected to the third receiving preamplifier is arranged. FIG. 11C is a graph illustrating the receiving sensitivity characteristics of the element group 210 having the above-described arrangement. In figures illustrating the characteristics, characteristics of one element at the center, an average of characteristics of 6 first ultrasonic transducer elements surrounding the center element, and an average of characteristics of 12 second ultrasonic transducer elements surrounding the first ultrasonic transducer elements are illustrated.

The frequency characteristics illustrated in FIG. 11C are those of a voltage signal which is obtained by amplification and conversion of the output current generated from the received ultrasonic wave in the element group 210. The amplification and conversion are executed by each receiving preamplifier 208. At this time, a distance between a center of the element group 210 and a point sound source 204 is 100 mm, and a spherical wave is generated by the point sound source 204. A value on an axis of ordinates is normalized by a peak value of the receiving sensitivity of the element group 210 disposed at the center.

In FIG. 11C, “center element” indicates characteristics of the element 201 arranged at a center (center element), and “first elements surrounding center element” indicates characteristics of the ultrasonic transducer elements 201 disposed around the center element (first ultrasonic transducer elements). Further, “second elements surrounding center element” indicates characteristics of the ultrasonic transducer elements 201 disposed around the first ultrasonic transducer elements surrounding the center element. The current/voltage conversion gains of the elements 201 in the element group 210 are different from each other according to the arrangement positions of the elements 201. Therefore, even when the sensitivity is lowered due to the directionality, the receiving sensitivity can be increased higher than the receiving sensitivity in the first exemplary embodiment. Particularly, the receiving sensitivity can be enhanced at a frequency higher than a frequency at which the peak sensitivity of the center element appears. When the receiving sensitivities of the center, first, and second ultrasonic transducer elements 201 at 10 MHz are compared with each other, the receiving sensitivity of the first ultrasonic transducer elements surrounding the center element is approximately 2% higher than the receiving sensitivity of the center element. The receiving sensitivity of the second ultrasonic transducer elements surrounding the first ultrasonic transducer elements is approximately 10% lower than the receiving sensitivity of the center element. The receiving sensitivities on a side of the high frequency of the first and second ultrasonic transducer elements surrounding the center element are improved more than the receiving sensitivity in the first exemplary embodiment. The sensitivity characteristics of the element arranged away from the center of the sensor plane 181 is enhanced higher than the sensitivity characteristics of the element arranged at the center of the sensor plane 181 so that the sensitivity of the ultrasonic transducer can be improved. Further, in order to improve the sensitivity of the ultrasonic transducer, the sensitivity characteristics of the elements arranged away from the center of the sensor plane are made higher as a distance of each of the elements from the center increases.

In other words, it is possible to prevent decrease in resolution due to the directionality of the ultrasonic transducer facing away from the center of curvature of the hemispherical acoustic wave sensor, and to achieve the mounting of ultrasonic transducers with high density while deterioration of the image quality due to resolution distribution within an image field of view is prevented.

A third exemplary embodiment will be described. In the present exemplary embodiment, the sensitivity is increased by changing a circuit constant and a spring constant of the device. Similar to the second exemplary embodiment, in order to enhance the detection sensitivity to the acoustic wave generated at the center 182 of curvature, the feedback resistance value and the feedback capacitance value of the receiving preamplifier 208 and a spring constant of the elements are changed. There are prepared three types of combinations of the feedback resistance value and the feedback capacitance value of the receiving preamplifier 208 to be connected to the ultrasonic transducer element 201. In the first combination, the feedback resistance value is 3480Ω, and the feedback capacitance value is 15 pF. In the second combination, the feedback resistance value is 4320Ω, and the feedback capacitance value is 15 pF. In the third combination, the feedback resistance value is 6040Ω, and the feedback capacitance value is 12 pF. Further, there are prepared three types of thicknesses of the fourth insulation layer 8 (sealing film). The first thickness is 450 nm, the second thickness is 650 nm, and the third thickness is 850 nm. The parameters and constructions other than the above are the same as those of the first exemplary embodiment. The ultrasonic transducer 200 and the ultrasonic unit 180 can be fabricated by the same method as in the first exemplary embodiment.

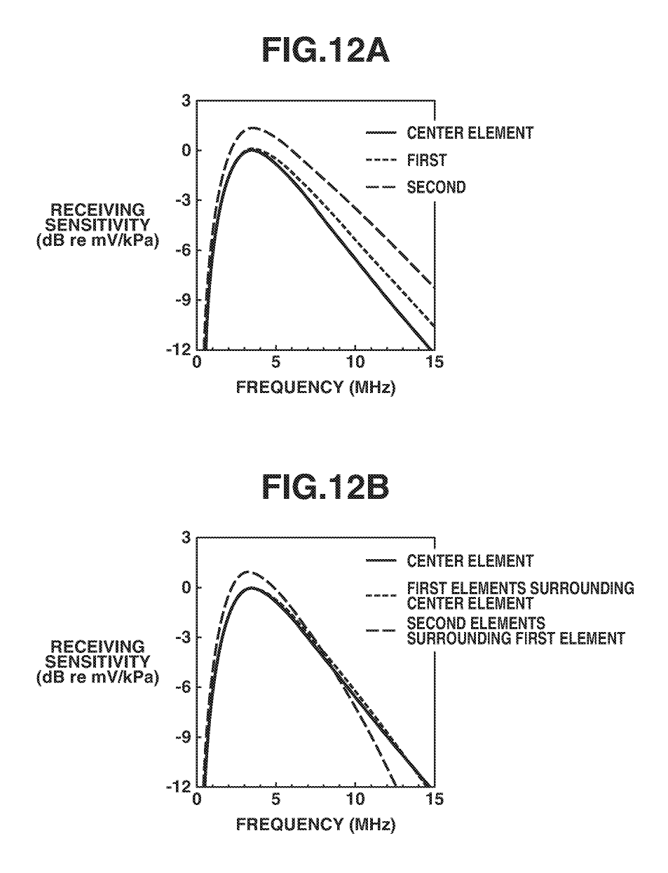

FIG. 12A is a graph illustrating the frequency characteristics of the receiving sensitivity in the cases where three types of the receiving preamplifiers 208 are respectively connected to three types of the element 201. A distance between a center of the element 201 and a point sound source 204 is 100 mm, and a spherical wave is generated at the point sound source 204. The characteristics are frequency characteristics of a voltage signal which is obtained by amplification and conversion of the output current generated from the received ultrasonic wave in each element 201. The amplification and conversion are executed by each receiving preamplifier 208. The output current characteristics of three types of the elements 201 are different from each other since the spring constants are different from each other. The element with the first sealing film having a thickness of 450 nm is connected to the first receiving preamplifier, the element with the second sealing film having a thickness of 650 nm is connected to the second receiving preamplifier, and the element with the third sealing film having a thickness of 850 nm is connected to the third receiving preamplifier. A value on an axis of ordinates is normalized by a peak value of the receiving sensitivity of the element 201 connected the first receiving preamplifier.

In FIG. 12A, “center element” indicates the receiving sensitivity characteristics of the element connected to the first receiving preamplifier, and “first” indicates the receiving sensitivity characteristics of the element connected to the second receiving preamplifier. Further, “second” indicates the receiving sensitivity characteristics of the element connected to the third receiving preamplifier. By changing the spring constant of the element 201 and the current/voltage conversion gain of the receiving preamplifier 208, the receiving sensitivity of the first elements is made larger than that of the center element, and the receiving sensitivity of the second elements is made larger than that of the first elements. At the center illustrated in FIG. 6A, the element 201 having such receiving sensitivity characteristics connected to the first receiving preamplifier is arranged. For the first elements surrounding the center element, the elements 201 connected to the second receiving preamplifier are arranged. For the second elements surrounding the first elements, the elements 201 connected to the third receiving preamplifier are arranged. FIG. 12B is a graph illustrating the receiving sensitivity characteristics of the element group 210 with the above-described arrangement.

The frequency characteristics illustrated in FIG. 12B is frequency characteristics of a voltage signal which is obtained by amplification and conversion of the output current generated from the received ultrasonic wave in each element group 210. The amplification and conversion are executed by each receiving preamplifier 208. At this time, a distance between a center of the element group 210 and a point sound source 204 is 100 mm, and a spherical wave is generated by the point sound source 204. A value on an axis of ordinates is normalized by a peak value of the receiving sensitivity of the element group 210 disposed at the center.

In FIG. 12B, “center element” indicates characteristics of the element 201 arranged at the center (center element), and “first elements surrounding center element” indicates characteristics of the elements 201 arranged around the center element 201 (first elements). Further, “second elements surrounding first element” indicates characteristics of the elements 201 arranged around the first elements surrounding the center element. The current/voltage conversion gains and the spring constants of the elements 201 in the element group 210 are different from each other according to the arrangement positions of the elements 201. Therefore, even when the receiving sensitivity is lowered due to the directionality, the receiving sensitivity can be increased higher than the receiving sensitivity in the first exemplary embodiment. When the receiving sensitivities at 10 MHz of the center, first, and second elements are compared with each other, the receiving sensitivity of the first elements surrounding the center element is approximately 4% higher than the receiving sensitivity of the center element. The receiving sensitivity of the second elements surrounding the first elements is approximately 7% lower than the receiving sensitivity of the center element. The receiving sensitivities of the first and second elements surrounding the center element are improved more than those of the first exemplary embodiment. The sensitivity characteristics of the elements arranged away from the center of the sensor plane 181 is enhanced higher than the sensitivity characteristics of the element arranged at the center of the sensor plane 181. Hence, the sensitivity of the ultrasonic transducer 200 can be improved. Further, in order to improve the sensitivity of the ultrasonic transducer 200, the sensitivity characteristics of the elements arranged away from the center of the sensor plane 181 are made higher as a distance of each of the elements from the center increases.

In other words, it is possible to prevent decrease in resolution due to the directionality of the ultrasonic transducer facing away from the center of curvature of the hemispherical acoustic wave sensor, and to achieve the mounting of the ultrasonic transducers with high density while deterioration of the image quality due to resolution distribution within an image field of view is prevented.

A fourth exemplary embodiment will be described. In the present exemplary embodiment, a size of the elements arranged around the center element is decreased. Similar to the second and third exemplary embodiments, in order to enhance the detection sensitivity to the acoustic wave generated at the center 182 of curvature, the size of the element is changed, and the combination of the feedback resistance value and the feedback capacitance value of the receiving preamplifier 208 to be connected to the element is changed. There are prepared three types of sizes of the element. The first element has a diameter of 2 mm, the second element has a diameter of 1.5 mm, and the third element has a diameter of 1.2 mm. The number of cells in the first element is 2400, the number of cells in the second element is 1340, and the number of cells in the third element is 850. The capacitance value of the first element is 125 pF, that of the second element is 75 pF, and that of the third element is 54 pF. Further, there are prepared three types of receiving preamplifiers 208. In the first combination of the feedback resistance value and the feedback capacitance value, the feedback resistance value is 3480Ω, and the feedback capacitance value is 15 pF. In the second combination, the feedback resistance value is 6040Ω, and the feedback capacitance value is 8 pF. In the third combination, the feedback resistance value is 9760Ω, and the feedback capacitance value is 4 pF.

As illustrated in FIG. 6A, an interval p1 between the center and the first elements surrounding the center element is 2 mm, and an interval p2 between the center and the second elements surrounding first elements is 3.45 mm. The capacitive transducer element groups 210 as illustrated in FIG. 6A are arranged. The parameters and configurations other than the above are the same as those of the first exemplary embodiment. The ultrasonic transducer 200 including the element groups 210 and the ultrasonic unit 180 can be fabricated by the same method as in the first exemplary embodiment.

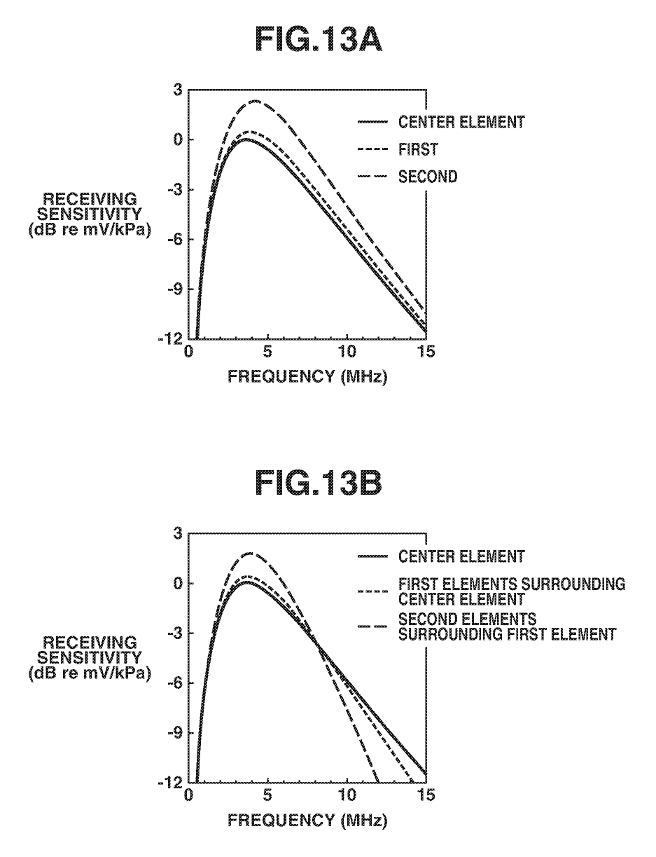

FIG. 13A is a graph illustrating the frequency characteristics of the receiving sensitivity in the cases where three types of the receiving preamplifiers 208 are respectively connected to the three types of the elements 201. The distance between a center of the element 201 and a point sound source 204 is 100 mm, and a spherical wave is generated at the point sound source 204. The frequency characteristics illustrated in FIG. 13A is frequency characteristics of a voltage signal which is obtained by amplification and conversion of the output current generated from the received ultrasonic wave in each element 201. The amplification and conversion are executed by each receiving preamplifier 208. The output current characteristics of three types of the elements 201 are different from each other since the sizes of the elements are different from each other. The element having the first diameter of 2 mm is connected to the first receiving preamplifier, the element having the second diameter of 1.5 mm is connected to the second receiving preamplifier, and the element having the third diameter of 1.2 mm is connected to the third receiving preamplifier. A value on an axis of ordinates is normalized by a peak value of the receiving sensitivity of the element 201 connected the first receiving preamplifier.

In FIG. 13A, “center element” indicates the receiving sensitivity characteristics of the element 201 connected to the first receiving preamplifier, and “first” represents the receiving sensitivity characteristics of the element 201 connected to the second receiving preamplifier. Further, “second” represents the receiving sensitivity characteristics of the element connected to the third receiving preamplifier. By changing the size of the element 201 and the current/voltage conversion gain of the receiving preamplifier, the receiving sensitivity of the first element is made larger than that of the center element, and the receiving sensitivity of the second element is made larger than that of the first element. At the center illustrated in FIG. 6A, the element 201 having receiving sensitivity characteristics of the element connected to the first receiving preamplifier is arranged. For the first elements surrounding the center element, the elements 201 connected to the second receiving preamplifier are arranged. For the second elements surrounding the first elements, the elements 201 connected to the third receiving preamplifier are arranged. FIG. 13B is a graph illustrating the receiving sensitivity characteristics of the element group 210 having the above-described arrangement.

The frequency characteristics illustrated in FIG. 13B is frequency characteristics of a voltage signal which is obtained by amplification and conversion of the output current generated from the received ultrasonic wave in each element group 210. The amplification and conversion are executed by each receiving preamplifier 208. At this time, a distance between a center of the element group 210 and a point sound source 204 is 100 mm, and a spherical wave is generated by the point sound source 204. A value on an axis of ordinates is normalized by a peak value of the receiving sensitivity of the element group 210 disposed at the center.

In FIG. 13B, “center element” indicates characteristics of the center element 201 arranged at the center, and “first elements surrounding center element” indicates characteristics of the first elements 201 surrounding the center element 201. Further, “second elements surrounding center element” indicates characteristics of the second elements 201 surrounding the first elements 201. The sizes and current/voltage conversion gains of the elements 201 in the element group 210 are different from each other according to the arrangement position. Therefore, even when the receiving sensitivity is lowered due to the directionality, the receiving sensitivity can be higher than the receiving sensitivity in the first exemplary embodiment. When the receiving sensitivities at 10 MHz are compared with each other, the receiving sensitivity of the first elements 201 surrounding the center element 201 is approximately 4% lower than the receiving sensitivity of the center element 201. The receiving sensitivity of the second elements 201 surrounding the first elements 201 is approximately 18% lower than the receiving sensitivity of the center element 201. The receiving sensitivities of the first and second elements 201 surrounding the center element 201 are improved. The sensitivity characteristics of the element arranged away from the center of the sensor plane 181 is enhanced higher than the sensitivity characteristics of the element arranged at the center of the sensor plane 181. Hence, the sensitivity of the ultrasonic transducer 200 can be improved. Further, in order to improve the sensitivity of the ultrasonic transducer 200, the sensitivity characteristics of the elements arranged away from the center of the sensor plane 181 is made higher as a distance of each of the elements from the center increases.

In other words, it is possible to prevent decrease in resolution due to the directionality of the ultrasonic transducer facing away from the center of curvature of the hemispherical acoustic wave sensor, and to achieve the mounting of the ultrasonic transducers with high density while deterioration of the image quality due to a resolution distribution within an image field of view is prevented.

According to aspects of the present invention, there are arranged a ultrasonic transducer element facing approximately a center of curvature of a probe casing, and a ultrasonic transducer element facing away from the center of curvature of the probe casing. Hence, a resolution distribution within a region of an inspection target (subject) can be improved.

While aspects of the present invention have been described with reference to exemplary embodiments, it is to be understood that aspects of the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-045038, filed Mar. 8, 2016, which is hereby incorporated by reference herein in its entirety.

What is claimed is:

1. An ultrasonic transducer unit comprising:
an ultrasonic transducer including a plurality of ultrasonic transducer elements; and
a probe casing configured to support a plurality of the ultrasonic transducers, and to have a concave portion facing a subject to be located at a predetermined position,
wherein the plurality of ultrasonic transducer elements is arranged on a same plane facing a center of curvature of the probe casing, and
wherein the plurality of ultrasonic transducer elements is arranged in a rotationally symmetrical manner about a normal line connecting the center of curvature of the probe casing to a point on a plane of the ultrasonic transducer.

2. The ultrasonic transducer unit according to claim 1, wherein the plurality of ultrasonic transducer elements is arranged about the normal line in such a manner that an arrangement of the plurality of ultrasonic transducer elements when the plane is rotated about the normal line by an angle excluding 360 degree remains unchanged from an original arrangement.

3. The ultrasonic transducer unit according to claim 2, wherein the plurality of ultrasonic transducer elements is arranged about the normal line in such a manner that an arrangement of the plurality of ultrasonic transducer elements every time when the plane is rotated about the normal line at an equi-angular interval remains unchanged from the original arrangement.

4. The ultrasonic transducer unit according to claim 1, wherein a center ultrasonic transducer element of the plurality of ultrasonic transducer elements is disposed at a position, on the plane, through which the normal line passes.

5. The ultrasonic transducer unit according to claim 4, wherein the plurality of ultrasonic transducer elements is arranged about the center ultrasonic transducer element, which is disposed at the position through which the normal line passes, in a manner such that the plurality of ultrasonic transducer elements is arranged in more than one concentric circle.

6. The ultrasonic transducer unit according to claim 1, wherein no ultrasonic transducer element is disposed at a position, on the plane, through which the normal line passes.

7. The ultrasonic transducer unit according to claim 1, wherein the plurality of ultrasonic transducer elements includes an ultrasonic transducer element arranged facing the center of curvature of the probe casing, and an ultrasonic transducer element arranged facing away from the center of curvature of the probe casing.

8. The ultrasonic transducer unit according to claim 1, wherein a sensitivity of an ultrasonic transducer element disposed away from a center of the plane is higher than a sensitivity of an ultrasonic transducer element disposed closer to the center of the plane than the ultrasonic transducer element disposed away from the center of the plane.

9. The ultrasonic transducer unit according to claim 1, wherein a size of an ultrasonic transducer element disposed away from a center of the plane is smaller than a size of an ultrasonic transducer element disposed closer to the center of the plane than the ultrasonic transducer element disposed away from the center of the plane.

10. The ultrasonic transducer unit according to claim 1, wherein the plurality of the ultrasonic transducers is arranged at an approximately equal interval along a generating line connecting a point at an upper edge of the concave portion of the probe casing to a point at a bottom center of the concave portion, and an arrangement of the plurality of the ultrasonic transducers is repeated at an equi-angular interval about the bottom center.

11. The ultrasonic transducer unit according to claim 1, wherein the ultrasonic transducer element is a capacitive transducer element including a plurality of cells in each of which a vibration film having one of a pair of electrodes which is formed in a vibratory manner with a space therebetween.

12. An information acquisition apparatus comprising:
the ultrasonic transducer unit according to claim 1; and
a processing portion,
wherein the ultrasonic transducer unit detects an acoustic wave from the subject, and outputs a detection signal, and
wherein the processing portion processes the detection signal to acquire information of the subject.

13. An information acquisition apparatus comprising:
the ultrasonic transducer unit according to claim 1;
a light source configured to emit light; and
a processing portion,
wherein the ultrasonic transducer unit detects a photoacoustic wave generated in the subject irradiated with the light emitted by the light source, and outputs a detection signal, and
wherein the processing portion processes the detection signal to acquire information of the subject.

14. The information acquisition apparatus according to claim 12, further comprising:
a display portion,
wherein the processing portion processes the detection signal to acquire image information of the subject, and
wherein the display portion displays an image of the subject based on the image information.
